(19) United States
(12) Patent Application Publication
(10) Pub. No.: US 2008/0051750 A1
(43) Pub. Date: Feb. 28, 2008

(54) Array of feminine hygiene products

(76) Inventors: Stephanie Schagen, Berlin (DE); Julien Edouard Levy, Geneva (CH); Annemarie Elizabeth Josephine Adriaanse, Pelia-Lancyl (CH); Kristof Neirynck, Geneva (CH)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/509,496

(22) Filed: Aug. 24, 2006

(51) Int. Cl. A61F 13/15

(52) U.S. Cl. 604/385.02

(57) ABSTRACT

An array of absorbent article products including a first absorbent article product and a second absorbent article product. The first absorbent article product includes a first absorbent article having a first benefit and is at least partially enclosed in a first package. The first absorbent article product further includes first indicia and second indicia. At least a portion of the first indicia corresponds to a first feminine aspirational state. The second absorbent article product includes a second absorbent article having a second benefit and is at least partially enclosed in a second package. The second absorbent article product further includes third indicia and fourth indicia. At least a portion of the third indicia corresponds to a second feminine aspirational state and the fourth indicia relate to the second indicia.

### FIELD OF THE INVENTION

The present invention relates generally to the field of absorbent feminine hygiene articles, such as, for example sanitary napkins, panty liners and adult incontinence articles.

### BACKGROUND OF THE INVENTION

Sanitary napkins for absorbing and handling body exudates, such as menses or urine, are widely known in the art. Equally well known are sanitary napkins enclosed in a package having indicia. Indicia on a package of sanitary napkins can come in a wide variety of forms and can perform a wide variety of functions. The indicia may take the form of words, shapes, colors, pictures, symbols and the like. Oftentimes, the indicia are used to increase a consumer's awareness of a particular attribute of the sanitary napkins, such as improved absorbency. In other instances, the indicia may used to identify the manufacturer of the sanitary napkins. In yet other examples, the indicia may be used to communicate a particular message concerning the quality of the sanitary napkins or the consumer's need for the sanitary napkins. In still yet other examples, the indicia may be used to communicate how satisfied the consumer will be if she were to purchase the sanitary napkins.

A manufacturer or retailer of sanitary napkins may go to great lengths to communicate to a consumer the reason(s) why the consumer should purchase its sanitary napkins rather than the sanitary napkins of a competitor. The methods of communication employed may be subtle, such as, for example consumer preferred colors on a package. On the other hand, the communication may be very direct, such as, for example boldly depicted words that spell out the manufacturer's message.

One example includes a package of sanitary napkins having indicia capable of communicating to a consumer the idea that the sanitary napkins contained within the package provide a superior benefit as compared to the products of one or more competitors. This approach is commonly known as comparative advertising. Unfortunately, this approach has several drawbacks. For instance, the claims must typically be supported by appropriate data that can often be time consuming and expensive to generate. Another drawback is that a superiority claim may only apply to one of several important attributes desired by the consumer. Thus, even if the manufacturer is successful in convincing consumers that its sanitary napkins are superior with regard to the attribute to which the superiority claims are made, many consumers may still have the impression that other attributes of the article are inferior to other articles in the market. Yet another drawback is that comparative claims, while possibly significant to one of technical skill in the art, may not address the target consumer's desired information about the product in a way that is useful for the consumer or provided in a way that advantageously makes a connection with the consumer that will result in a sale of the product. For example, it may be important to provide information about a sanitary napkin in a way that appeals to a woman's sense of femininity in addition to or as opposed to a mere recitation of the technical benefits of a particular product. One more downside to typical comparative and/or technical advertising is that it often fails to give the consumer an overall impression with respect to a particular product lineup or related array of articles.

One approach to overcome the above problems is to have indicia capable of communicating to a woman a feminine aspirational state she may wish to experience by using a particular feminine hygiene product or a particular sanitary napkin. For example, a manufacturer of sanitary napkins may wish to use a feminine aspirational image to communicate to a woman the feminine aspirational state of reliable or ultimate protection from a sanitary napkin. In another example, a manufacturer or retailer of feminine hygiene products may wish to include one or more feminine aspirational images on a package of feminine hygiene products or other media associated with one or more feminine hygiene products to communicate to a user the idea that the feminine hygiene products can be used for feminine beauty enhancement. The association of the feminine hygiene products with the concept of feminine beauty enhancement, as opposed to a more typical consumer view that feminine hygiene products are merely sanitary or medicinal devices, may result in increased sales.

Thus, it may be desirable to provide a package or other media for feminine hygiene articles that include indicia that communicate a benefit associated with the feminine hygiene article to a user. It may also be desirable to have a feminine aspirational state associated with the benefit. It may further be desirable to have a feminine aspirational image associated with one or more feminine aspirational states. It may still further be desirable to provide indicia on an array of feminine hygiene products or media associated therewith that relate the particular feminine hygiene products to each other. It may still even further be desirable to provide indicia on an array of feminine hygiene product packages or a media associated therewith that relate the feminine hygiene products to the concept of feminine beauty enhancement.

### SUMMARY OF THE INVENTION

In order to provide a solution to the inconveniences set forth above, one embodiment of the present invention provides an array of absorbent products. The array of absorbent article products comprises a first absorbent article product and a second absorbent article product. The first absorbent article product may include a first absorbent article having a first benefit and is at least partially enclosed in a first package. The first absorbent article product may further include first indicia and second indicia. At least a portion of the first indicia may correspond to a first feminine aspirational state. The second absorbent article product may include a second absorbent article having a second benefit and is at least partially enclosed in a second package. The second absorbent article product may further include third indicia and fourth indicia. At least a portion of the third indicia may correspond to a second feminine aspirational state and the fourth indicia may relate to the second indicia.

### BRIEF DESCRIPTION OF THE DRAWINGS

FIGS. 1A-1D show an array of absorbent article products according to one embodiment of the present invention.

FIGS. 2A-2D show an array of absorbent article products according to one embodiment of the present invention.

FIG. 3 shows an example of a depiction of a feminine aspirational image according to the present invention.

FIG. 4 shows an example of a depiction of a feminine aspirational image according to the present invention.

FIG. 5 shows an example of a depiction of a feminine aspirational image according to the present invention.

FIG. 6 shows an example of a depiction of a feminine aspirational image according to the present invention.

### DETAILED DESCRIPTION OF THE INVENTION

As noted above, current promotion and advertising of feminine hygiene products may be inadequate or at least not fully effective due to the technical nature of the advertising and/or the lack of association of the benefits of the product with feminine beauty enhancement and/or feminine aspirational states. Accordingly, one approach that may change a consumer's perception of feminine hygiene products and the desire to purchase such products includes associating the feminine hygiene product itself with the concept of feminine beauty and beauty enhancement.

One way to promote the association of feminine hygiene products to feminine beauty is to provide a feminine hygiene product or an array of feminine hygiene products that include packaging or other advertising indicia capable of communicating to a consumer the idea that the feminine hygiene product or the array of feminine hygiene products are a beauty care item as opposed to strictly a hygienic device. One example of such indicia includes indicia that correspond to one or more feminine aspirational states. The feminine aspirational states associated with a product or an array of products may communicate to a consumer the idea that feminine hygiene products are no longer simply functional feminine hygiene products, but are beauty care items that provide a woman with choices on how to enhance particular facets of her feminine beauty.

As used herein the term “feminine aspirational state” generally refers to a state that a woman may desire to achieve through use of a product that offers a particular benefit or feature. The feminine aspiration state may be physical in nature, such as, for example a state of physical cleanliness. Alternatively, the feminine aspirational state may be emotional in nature, such as, for example a state of feeling secure or protected. Further, the feminine aspirational state may be aesthetic in nature, such as, for example a state of being beautiful. The above mentioned feminine aspirational states and those described hereinbelow are merely exemplary in nature, and are not intended to be limiting. Accordingly, it should be recognized that a feminine aspiration state may include any one or more of those described herein or any other relevant feminine aspiration state.

During her period a woman may experience undesirable feelings, such as, for example a feeling of insecurity related to the performance of her feminine hygiene product, such as, for example her sanitary napkin or tampon. A feeling of insecurity may arise, for example, from the fact that the woman is using an inferior feminine hygiene product or that the woman is experiencing heavier menstrual flow than usual and is unsure as to whether her feminine hygiene product will provide adequate protection. The feeling of insecurity related to feminine hygiene product use might not be as strong if the user had some reassurance that the feminine hygiene product would provide the desired level of protection.

One way to provide such reassurance is to provide indicia on the feminine hygiene product package or other media associated with the product that can communicate to a user a feminine aspirational state associated with security. The positive feelings a user may associate with the feminine aspirational state of security provided by the indicia can allow a user to select one particular feminine hygiene product out of an array of products to enhance her feminine beauty. For example, a user may wish to wear a particular outfit, but because she is having her period, does not feel secure enough to wear it. This feeling of insecurity might be analogous to the feeling of insecurity a user may experience if she feels she does not have the proper make-up or shoes to wear with a particular outfit. Thus, by selecting the proper feminine hygiene product, the user may feel secure enough to wear the outfit and therefore may feel she has enhanced her feminine beauty.

The adequacy of protection provided by a feminine hygiene product may not be the only property capable of causing anxiety in a user. Other properties, such as comfort, odor control or cleanliness may also cause needless anxiety, and therefore, a consumer may desire a feminine hygiene product that provides a specific benefit tailored to the consumer's specific need or desired state of comfort related to the user's anxiety. One way to address this need is to provide a lineup of products that all share one or more fundamental benefits with the other products in the lineup, but wherein the products also provide one or more differential benefits that may be desired by a consumer. A differential benefit is a benefit provided by one product in a lineup that distinguishes the one product from the other products in the lineup. For example, a lineup of four sanitary napkins products may have differential benefits of thickness, softness, absorbency and odor control, respectively. A fundamental benefit, on the other hand, is a benefit that is generally provided by all members of the product lineup. Examples of fundamental benefits include, but are not limited to absorbent capacity, softness, wicking ability, fastening means that allow the sanitary napkin to be joined to a user's panty, benefits related to fabrication materials, product shape, product look, combinations thereof and the like. Each member of the product lineup can have one or more differential benefits or combinations of differential benefits, and such differential benefits may include some or all of the fundamental benefits set forth herein, depending on the particular array of products being offered.

Often feminine hygiene products that share fundamental benefits with other feminine hygiene products may be marketed by a manufacturer or retailer of such products as part of single product lineup. While a single product lineup may include products of the same general type, it need not necessarily do so. For example, the present invention contemplates a product lineup including several different kinds of sanitary napkins, but product lineups wherein the products are not the same type, such as, for example a lineup including a mix of sanitary napkins, tampons, and/or panty liners is also contemplated by the present invention.

Unfortunately, a consumer may not associate one feminine hygiene product of a lineup to the lineup itself, or may not be aware that there even is a product lineup. Therefore, in such a situation, the consumer may likely be unaware of the existence of any shared fundamental benefit(s) between one or more feminine hygiene products. In order to address a lack of awareness or the potential therefore on the part of a consumer, a manufacturer may choose to spend money on a marketing campaign in order to increase the consumer's awareness with regard to the nature of the product relationship, i.e., the existence of a product lineup and shared fundamental benefits. Therefore, by providing an array of feminine hygiene product packages that are capable of communicating to a user that the product purchased may provide the specific benefit the user desires, and that products providing different benefits are all part of the same product lineup, the manufacturer may provide adequate reassurance to the user and increased commercial success of the product.

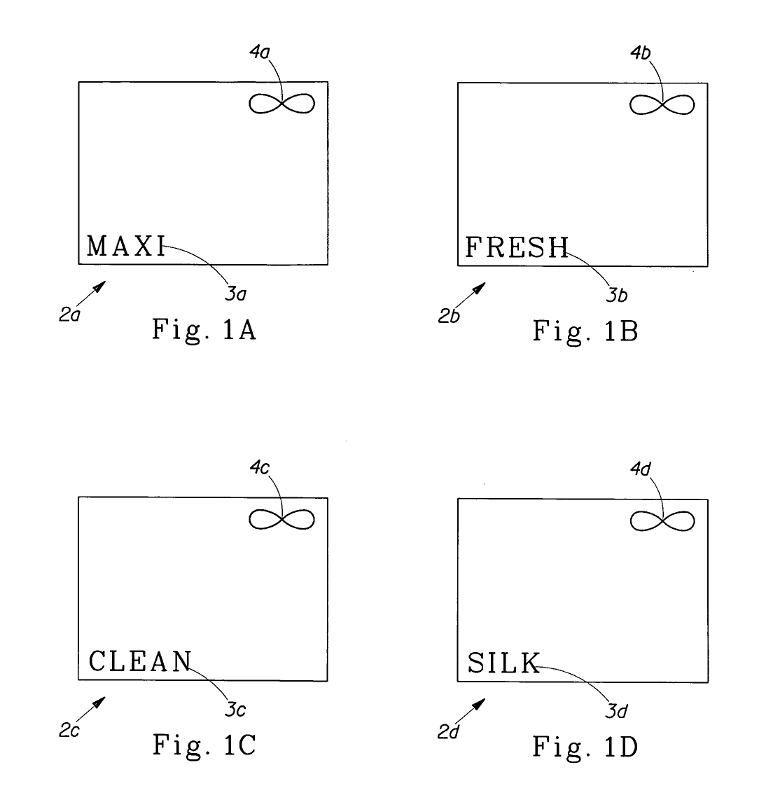

FIGS. 1A-1D show an example of one embodiment of the present invention including an array comprising four individual sanitary napkin packages 2a, 2b, 2c and 2d. Each package 2a, 2b, 2c and 2d has first indicia 3a, 3b, 3c and 3d and second indicia 4a, 4b, 4c and 4d. Any package known by those of ordinary skill in the art to be used for storing and/or dispensing sanitary napkins is suitable for use with the present invention. The sanitary napkins contained within a particular package 2a, 2b, 2c and 2d may provide one or more benefits that are different than the one or more benefits provided by the sanitary napkins contained in at least one other package 2a, 2b, 2c and 2d. By way of example and not limitation, the sanitary napkins contained in package 2a may provide the benefit of increased absorbency while the sanitary napkins in package 2b may provide the benefit of odor control. Alternatively, the one or more benefits provided by the sanitary napkins of one particular package 2a, 2b, 2c and 2d may be different than the one or more benefits provided by the sanitary napkins contained in all of the other packages 2a, 2b, 2c and 2d. One example of such an embodiment includes the sanitary napkins contained in packages 2a, 2b, 2c and 2d which provide the benefits of increased absorbency, odor control, cleanliness (which may include e.g. a personal cleansing wipe associated with or attached to the sanitary napkin or wrapper) and comfort, respectively.

The packages 2a, 2b, 2c and 2d of FIG. 1 include first indicia 3a, 3b, 3c and 3d which correspond to a benefit provided by the sanitary napkins contained in the package 2a, 2b, 2c and 2d. In order to provide assurance to a user that the user will receive the particular benefit, first indicia 3a, 3b, 3c, and 3d may also relate to a feminine aspirational state. In one example, the feminine aspirational state may be one where the user desires to feel secure in the knowledge that her sanitary napkin will provide adequate protection. Another example includes a feminine aspirational state includes a user's desires to feel clean, such as, for example the feeling of having just stepped out of the shower. Still other examples of feminine aspirational states include a user's desire to feel fresh or comfortable when using a particular sanitary napkin. In FIG. 1, the indicia 3a, 3b, 3c, and 3d include the terms “MAXI,” “FRESH,” “CLEAN” and SILK, respectively. Through advertising or other suitable means, a manufacturer may communicate to a consumer that these terms correspond to the feminine aspirational states of security, freshness, cleanliness and comfort. In the present example, the first indicia comprise words, however, it is to be understood that the first indicia may also comprise numbers, letters, symbols, shapes, images, colors, patterns, raised portions, combinations thereof or other suitable indicators capable of communicating. Although the present example describes an array including four packages 2a, 2b, 2c, and 2d, it is to be understood that an array comprising two or more packages are also contemplated by the present invention. Further, while the present examples may describe only four aspirational states, the present invention contemplates other embodiments including two or more aspirational states that may be the same or different than those listed herein.

When introducing a new line of products, a manufacturer may decide to introduce one product at a time, and consequently, may desire some way to communicate to a consumer that some or all of the products in the lineup are related. Alternatively, a manufacturer may wish to advertise one specific product out of a product lineup, but still have the consumer recognize the overall product relationship. To this end, the packages of FIG. 1 include second indicia 4a, 4b, 4c, and 4d. The second indicia 4a, 4b, 4c, and 4d may communicate to a consumer that all the products are from the same lineup. Thus, when a consumer is presented with the array of the present invention, she can tailor her feminine hygiene product selection based on her individual needs, and still understand that all products in the array share one or more fundamental benefits that she may desire.

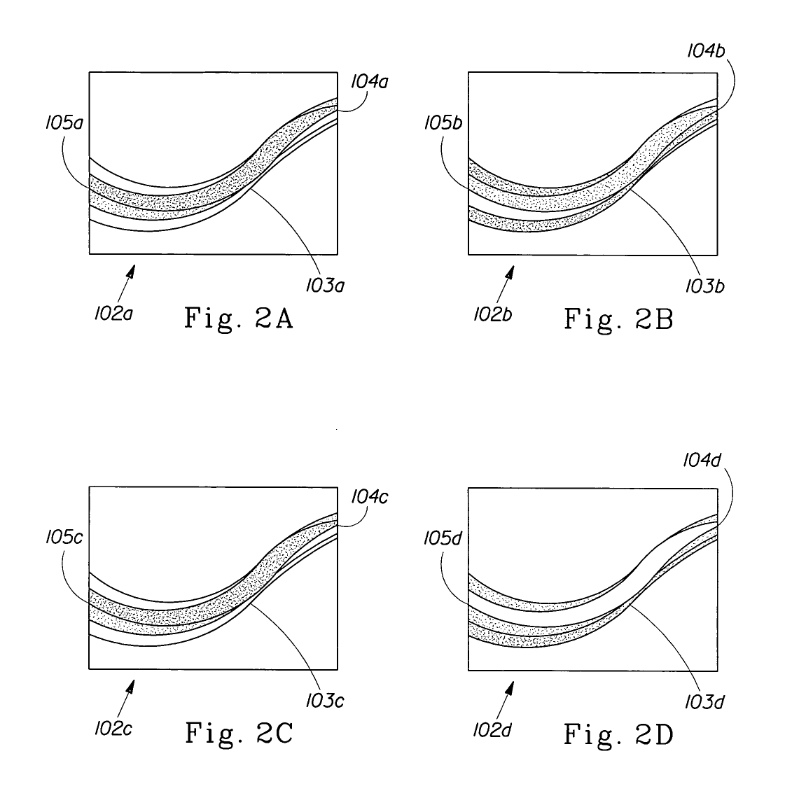

FIGS. 2A-2D show another example of an embodiment of the present invention including an array of feminine hygiene articles, such as, for example sanitary napkin packages 102a, 102b, 102c and 102d. Each package 102a, 102b, 102c and 102d has indicia 103a, 103b, 103c and 103d. The indicia 103a, 103b, 103c and 103d comprise a wave-like shape and have a narrow end 104a, 104b, 104c and 104d and a wide end 105a, 105b, 105c and 105d. The indicia 103a, 103b, 103c and 103d each may comprise different colors, combinations of different colors, different patterns or sequences of the same color, different shades of the same color, combinations of color and no color and combinations thereof. The sanitary napkins contained in the packages 102a, 102b, 102c and 102d may provide various differential benefits. Through advertising or other suitable means, a manufacturer may communicate to a consumer that indicia 103a, 103b, 103c and 103d each correspond to one or more particular feminine aspirational states, such as the feminine aspirational state of security, freshness, cleanliness and/or comfort. The manufacturer may further communicate to a consumer that the indicia 103a, 103b, 103c and 103d each correspond to one or more particular benefits provided by a sanitary napkin contained in a package 102a, 102b, 102c and 102d. Therefore, based on indicia 103a, 103b, 103c and 103d a consumer may be able to determine which package 102a, 102b, 102c and 102d contains the sanitary napkins that provide the benefit she desires, and which feminine aspirational state corresponds to the benefit.

In one embodiment, for example, a sanitary napkin package, such as package 102b, may include indicia 103b comprising four different shades of blue. The four different shades of blue may span a spectrum of light blue to dark blue and may be arranged in a particular pattern, such as, for example the pattern shown in FIG. 2B. A consumer may recognize the color pattern arrangement or the particular shades of blue as corresponding to a particular benefit provided by the sanitary napkin, such as, for example the benefit of high absorbent capacity. The consumer may further recognize from the indicia 103b that the benefit corresponds to a particular feminine aspirational state, such as, for example protection from leakage.

It can be seen from FIGS. 2A-2D that the similar or identical shapes of the indicia 103a, 103b, 103c and 103d as well as the similar way the color patterns are depicted may communicate to a consumer the idea that the products are all part of a single product lineup. For example, the wavelike shape of indicia 103a, 103b, 103c and 103d may provide a highly recognizable image, such that a consumer may associate the indicia 103a, 103b, 103c and 103d with a particular array of sanitary napkins, and therefore recognize that the products are all part of the same product lineup.

While the preceding example refers to the color pattern of indicia 103a, 103b, 103c and 103d as means to communicate to a consumer a product benefit and a feminine aspirational state, and the wavelike shape of indicia 103a, 103b, 103c and 103d a means to communicate to a consumer that the products are part of the same product lineup, it is to be understood that the present invention is not limited to such an embodiment. For example, the present invention also contemplates embodiments where the wavelike shape or any other suitable shape of the indicia 103a, 103b, 103c and 103d communicates to a consumer a product benefit and/or a feminine aspirational state, and the color pattern of indicia 103a, 103b, 103c and 103d communicates to a consumer that the products are part of the same product lineup. Other embodiments where either the shape and/or the color pattern of the indicia 103a, 103b, 103c and 103d communicate to a consumer a product benefit, a feminine aspirational state, and a common product lineup are also contemplated by the present invention.

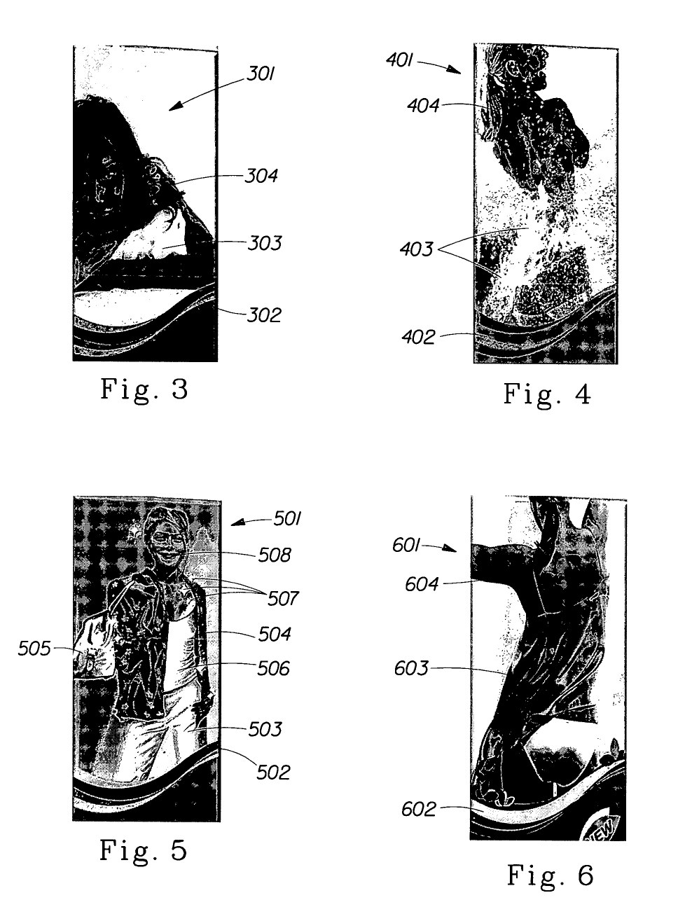

An alternative approach to communicating to a consumer what benefit or benefits is provided by a particular feminine hygiene product and which feminine aspirational state is associated with a particular benefit may include the use of a feminine aspirational image. As used herein the term “feminine aspirational image” generally refers to an image portraying a feminine aspirational state. In certain embodiments, the image may include a woman or a representation of a woman portraying the feminine aspirational state. By utilizing one or more feminine aspirational images, such as, for example the feminine aspirational images 301, 401, 501 and 601 of FIGS. 3, 4, 5, and 6, and indicia 302, 402, 502 and 602, a consumer may be able to determine which package 102a, 102b, 102c or 102d of FIGS. 2A-2D contain the sanitary napkins she desires to purchase. For example, a consumer who is unfamiliar with indicia 103a, 103b, 103c and 103d of FIGS. 2A-2D, but wishes to purchase sanitary napkins that provide suitable protection, need only observe the feminine aspirational image 501 and recognize that indicia 502 match indicia 103b in order to determine that package 102b of FIG. 2B contains the appropriate sanitary napkins.

The feminine aspirational image 301 of FIG. 3 depicts a woman 304 and a pillow 303. It is well known to those of ordinary skill in the art that, in the past, the level of protection provided by a sanitary napkin often corresponded to its thickness, i.e., the thicker the sanitary napkin, the more it could absorb, and thus, at least in theory, the better it would protect. Based on the notion that a thicker sanitary napkin provides better protection, many women may still find they feel more secure when using a thicker sanitary napkin. Therefore, a feminine aspirational image 301 that depicts a woman 304 lying on a thick pillow 303 in her own bed may illustrate a feminine aspirational state of security.

In addition to illustrating a feminine aspirational state, a manufacturer of sanitary napkins may also wish to include in the feminine aspirational image indicia that correspond to a particular sanitary napkin product. In the example illustrated by FIG. 3, indicia 302 may be identical to indicia 103a of FIG. 2A, and thus, a consumer who feels most secure when using a thick sanitary napkin may understand that sanitary napkin package 102a of FIG. 2A contains the appropriate sanitary napkins. While the indicia 302, 103a of the present example may be substantially the same in appearance they need not necessarily be so, and the present invention contemplates embodiments where the indicia 302, 103a may be similar, substantially similar, or otherwise related.

Similarly, FIG. 4 depicts feminine aspirational image 401 of a woman 404 being splashed with water 403. It is often assumed that a person is the cleanest when the person just finishes washing or showering. Thus, feminine aspirational image 401 may, for example, communicate the feminine aspirational state of cleanliness. Because feminine aspirational image 401 includes indicia 402 that match indicia 103c of package 102c of FIG. 2C, there is provided a means to communicate to a consumer who desires to purchase sanitary napkins that provide cleanliness, such as, for example a sanitary napkin having a wipe joined thereto or otherwise associated therewith, the message that package 102c of FIG. 2C contains the appropriate sanitary napkins.

FIG. 5 depicts feminine aspirational images 501, which may communicate to a consumer, for example, the feminine aspirational state of protection. A user may experience a feminine aspirational state of protection when she is using a sanitary napkin that provides a high absorbent capacity and/or absorbency rate. Typically, a woman might be unwilling to wear white or light colored clothing in a public place during her period because, should her sanitary napkin fail to absorb all of her menses, the resulting stain on the garment could be easily detected by others and could result in significant embarrassment. When a user is confident that her sanitary napkin is capable of effectively absorbing the volume of menstrual fluid she might expect to discharge during the time period she will use the product, the user may also be confident that she will not have to experience the potentially embarrassing situation of menstrual fluid becoming visible on her clothes as a result of non-absorption by the sanitary napkin. Because menses is typically more detectable on white or light colored garments than dark garments, the depiction of a confident woman wearing a substantially all-white or light colored garment on a package of sanitary napkins may communicate to a user that by using the product she will not face the potentially embarrassing situation described above. Feminine aspirational image 501 comprises a woman 504 dressed in a white shirt 506, white pants 503 and carrying a white purse 505. Disposed around the woman 504 are sparkles of light 507. The woman 504 depicted in FIG. 5 is dressed in a sparkling white outfit and is flashing a confident smile 508. A consumer may interpret the feminine aspirational image 501 to mean that the woman 504 is so confident that her sanitary napkin will provide the desired protection that she is willing to wear white clothing during her period. Further, the feminine aspirational image 501 includes indicia 502 that match the indicia 103b of sanitary napkin package 102b of FIG. 2B. Because feminine aspirational image 401 includes indicia 402 that match indicia 103c of package 102c of FIG. 2C, there is provided a means to communicate to a consumer who desires to purchase sanitary napkins that provide protection the message that package 102c contains the appropriate sanitary napkins.

FIG. 6 depicts feminine aspirational image 601 comprising a woman 604 wearing a silk garment 603. Silk is often associated with softness, and due to its softness is often used to make garments that are desired to be very soft and/or very comfortable. Thus, feminine aspirational image 601 may, for example, communicate the feminine aspirational state of ultimate comfort. Further, because the feminine aspirational image 601 includes indicia 602 that match the indicia 103d of package 102d of FIG. 2D, there is provided a means to communicate to a consumer who desires to purchase sanitary napkins that are soft and comfortable the message that package 102d contains the appropriate sanitary napkins.

A manufacturer or retailer desiring to communicate to a consumer the association of a particular feminine aspirational state with a particular feminine aspirational image may employ various marketing strategies. These marketing strategies can include, but are not limited to television ads, radio ads, hanging banners, mailers, shelf talkers, aisle displays, end caps, combinations thereof and the like. One particular strategy may be to provide advertising means, such as one or more hanging banners, located in at least one portion of a retail store. The one or more hanging banners may include at least one feminine aspirational image, such as, for example feminine aspirational image 301, that is capable of communicating the desired association to a consumer. A shopper who sees a hanging banner that includes, for example, feminine aspirational image 301 may then understand the relationship between the feminine aspirational image 301 and the feminine aspirational state of security and may realize that package 102a with similar indicia would provide the sanitary napkins with a differential benefit corresponding to security. If a plurality of hanging banners is desired, the banners may depict different feminine aspirational images, and may provide a means for identifying or selecting one or more particular products.

Yet another strategy of communicating to a consumer the association of a particular feminine aspirational state with a particular feminine aspirational image may be to have advertising means, such as, for example one or more shelf talkers or other visual display(s), in an area of a retail store where items such as make-up or lingerie are typically sold. Additionally, a manufacturer or retailer may desire to place advertising means containing different feminine aspirational images in different areas of a retail store. For example, a retailer or manufacturer may wish to place a feminine aspirational image, such as, for example feminine aspirational image 601, in an area of a retail store where lingerie is typically sold, while displaying a similar or different feminine aspirational image, such as, for example feminine aspirational image 401, in an area of the store where make-up is typically sold. By displaying the one or more feminine aspirational images in such a manner, the manufacturer may not only provide a means of communicating the relationship of the feminine aspirational state to the feminine aspirational image, but may also further reinforce the idea that the feminine hygiene product is a beauty care item. Further, one or more feminine hygiene products of a feminine hygiene product lineup, wherein each feminine hygiene product provides one or more differential benefits corresponding to one or more feminine aspirational states and/or feminine aspirational images, may be located in the same general area as the advertising means depicting the associated feminine aspirational image(s), but need not necessarily be so.

Still another method of communicating to a consumer a particular association between a feminine aspirational state and a feminine aspirational image might include combining different marketing strategies, or providing an overall marketing campaign. For example, a manufacturer may use television advertising to provide a more detailed explanation of a particular feminine aspirational state through the use of an actor who acts out the feminine aspirational state. The manufacturer may then display the acted-out feminine aspirational state in a retail environment through the use of an advertising means, such as, for example a shelf talker(s), display(s) or hanging banner(s). By using multiple advertising means, such as, for example television advertising and retail displays, a manufacturer may be able to communicate to a consumer that the product lineup includes fundamental benefits associated with all the products as well as differential benefits without having to advertise each product in the lineup separately.

Another method of communicating to a consumer a particular association between a feminine aspirational state and a feminine aspirational image may be to include one or more feminine aspirational images on the feminine hygiene product packages. Including one or more feminine aspirational images on a feminine hygiene product package may provide a direct means of communicating one or more particular feminine aspirational states to a consumer. In one embodiment, a package may contain a plurality of feminine hygiene products wherein at least one feminine hygiene product in the package provides a different benefit from at least one other feminine hygiene product in the package. In such instances, it may be desirable to include two or more different feminine aspirational images on the feminine hygiene product package and/or related materials. However, it should be understood that two or more aspirational images may be included on any package or related materials, including those that only include or relate to a single type or form of feminine hygiene product.

As used herein the term “joined” refers to the condition where a first member or component is attached, affixed, or otherwise physically connected to a second member or component either directly or indirectly. An example of an indirectly joined member or component might be where the first member or component is affixed, or connected to an intermediate member or component which in turn is affixed or connected to the second member or component.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

What is claimed is:

1. An array of absorbent article products, the array comprising:
a first absorbent article product, the first absorbent article product including a first package, a first absorbent article having a first benefit disposed at least partially within the first package, first indicia and second indicia, wherein at least a portion of the first indicia correspond to a first feminine aspirational state; and
a second absorbent article product, the second absorbent article product including a second package, a second absorbent article having a second benefit disposed at least partially within the second package, third indicia and fourth indicia, wherein at least a portion of the third indicia correspond to a second feminine aspirational state and the fourth indicia relate to the second indicia.

2. The array of claim 1, further comprising a third absorbent article product, the third absorbent article product including a third package, a third absorbent article having a third benefit at least partially disposed within the third package, fifth indicia and sixth indicia, wherein at least a portion of the fifth indicia correspond to a third feminine aspirational state and the sixth indicia relate to the second and fourth indicia.

3. The array of claim 2, further comprising a fourth absorbent article product, the fourth absorbent article product including a fourth package, a fourth absorbent article having a fourth benefit disposed at least partially within the fourth package, seventh indicia and eighth indicia, wherein at least a portion of the seventh indicia correspond to a fourth feminine aspirational state and the eighth indicia relate to the second, fourth and sixth indicia.

4. The array of claim 1, wherein the first feminine aspirational state is communicated to a user by an advertising means including a first feminine aspirational image.

5. The array of claim 4, wherein the second feminine aspirational state is communicated to a user by an advertising means including a second feminine aspirational image.

6. The array of claim 1, wherein the first and second absorbent articles include a fundamental benefit.

7. The array of claim 1, wherein the first and second benefits are differential benefits.

8. The array of claim 1, wherein the first and/or second feminine aspirational states include one or more of the following: freshness, cleanliness, softness, comfort, dryness, silkiness and combinations thereof.

9. The array of claim 1, wherein the first or second indicia is disposed on the first absorbent article and/or the third or fourth indicia is disposed on the second absorbent article.

10. The array of claim 1, wherein the first and/or second absorbent article include a wipe associated therewith.

11. An array of absorbent article products, the array comprising:
a first absorbent article having a first benefit, the first absorbent article being enclosed in a first package, the first package including first indicia and second indicia, wherein at least a portion of the first indicia correspond to a first feminine aspirational state; and
a second absorbent article having a second benefit, the second absorbent article being enclosed in a second package, the second package including third indicia and fourth indicia, wherein at least a portion of the second indicia correspond to a second feminine aspirational state and the fourth indicia relate to the second indicia.

12. The array of claim 11, wherein the first and second absorbent articles include a fundamental benefit.

13. The array of claim 11, wherein the first and second benefits are differential benefits.

14. The array of claim 11, wherein the first feminine aspirational state is communicated to a user by an advertising means including a first feminine aspirational image.

15. The array of claim 14, wherein the second feminine aspirational state is communicated to a user by an advertising means including a second feminine aspirational image.

16. The array of claim 11, wherein the first and/or second feminine aspirational states include one or more of the following: freshness, cleanliness, softness, comfort, dryness, silkiness and combinations thereof.

17. An array of absorbent article products, the array comprising:
a first absorbent article having a first benefit, the first absorbent article being enclosed in a first package, the first package including first indicia and second indicia, wherein the first indicia include one or more first feminine aspirational images corresponding to a first feminine aspirational state; and
a second absorbent article having a second benefit, the second absorbent article being enclosed in a second package, the second package including third indicia and fourth indicia, wherein the third indicia include one or more feminine aspirational images corresponding to a second feminine aspirational state and the fourth indicia relate to the second indicia.

18. The array of claim 17, wherein the first and second absorbent articles include a fundamental benefit.

19. The array of claim 17, wherein the first and second benefits are differential benefits.

20. The array of claim 17, wherein the first and/or second feminine aspirational states include one or more of the following: freshness, cleanliness, softness, comfort, dryness, silkiness and combinations thereof.
